(19) United States
(12) Patent Application Publication
(10) Pub. No.: US 2018/0273453 A1
(43) Pub. Date: Sep. 27, 2018

(54) PROCESS FOR THE PREPARATION OF GLYCOLS

(71) Applicant: SHELL OIL COMPANY, HOUSTON, TX (US)

(72) Inventors: Johannes Leo Marie VAN DER BIJL, Rijswijk (NL); Evert VAN DER HEIDE, Amsterdam (NL); Pieter HUIZENGA, Amsterdam (NL); Munro MACKAY, Amsterdam (NL)

(21) Appl. No.: 15/763,470

(22) PCT Filed: Sep. 27, 2016
(86) PCT No.: PCT/EP2016/073017

§ 371 Date: Mar. 27, 2018

Related U.S. Application Data
(60) Provisional application No. 62/234,122, filed on Sep. 29, 2015.

(51) Int. Cl. C07C 29/132; C07C 29/60; B01J 25/02; B01J 31/22; B01J 23/30

(52) U.S. Cl. CPC C07C 29/132; C07C 29/60; B01J 23/30; B01J 31/2239; B01J 25/02

(57) ABSTRACT

A process for the preparation of glycols from a saccharide-containing feedstock having the steps of: (a) preparing a reaction mixture in a reactor vessel comprising the saccharide-containing feedstock, a solvent, hydrogen, a catalyst component with retro-aldol catalytic capabilities and a first hydrogenation catalyst comprising an element selected from groups 8, 9 and 10 of the periodic table; (b) monitoring the hydrogenation activity in the reactor vessel; (c) when the activity of the first hydrogenation catalyst declines, as indicated by the crossing of a threshold, supplying into the reaction mixture in the reactor vessel a catalyst precursor comprising one or more elements selected from groups 8, 9, 10 and 11 of the periodic table; and (d) converting the catalyst precursor in the presence of hydrogen in the reactor vessel to a second hydrogenation catalyst to supplement the declined hydrogenation activity in the reactor vessel.

### FIELD OF THE INVENTION

The present invention relates to prolonging the hydrogenation activity of a process for the preparation of glycols from saccharide-containing feedstocks.

### BACKGROUND OF THE INVENTION

Glycols such as mono-ethylene glycol (MEG) and mono-propylene glycol (MPG) are valuable materials with a multitude of commercial applications, e.g. as heat transfer media, antifreeze, and precursors to polymers, such as PET. Ethylene and propylene glycols are typically made on an industrial scale by hydrolysis of the corresponding alkylene oxides, which are the oxidation products of ethylene and propylene, produced from fossil fuels.

In recent years, increased efforts have focussed on producing chemicals, including glycols, from non-petrochemical renewable feedstocks, such as sugar-based materials. The conversion of sugars to glycols can be seen as an efficient use of the starting materials with the oxygen atoms remaining intact in the desired product.

Current methods for the conversion of saccharides to glycols revolve around a two-step process of hydrogenolysis and hydrogenation, as described in Angew, Chem. Int. Ed. 2008, 47, 8510-8513.

Such two-step reaction requires at least two catalytic components. Patent application WO2015028398 describes a continuous process for the conversion of a saccharide-containing feedstock into glycols, in which substantially full conversion of the starting material and/or intermediates is achieved and in which the formation of by-products is reduced. In this process the saccharide-containing feedstock is contacted in a reactor vessel with a catalyst composition comprising at least two active catalytic components comprising, as a first active catalyst component with hydrogenation capabilities, one or more materials selected from transition metals from groups 8, 9 or 10 or compounds thereof, and, as a second active catalyst component with retro-aldol catalytic capabilities, one or more materials selected from tungsten, molybdenum and compounds and complexes thereof. Retro-aldol catalytic capabilities referred to herein means the ability of the second active catalyst component to break carbon-carbon bonds of sugars such as glucose to form retro-aldol fragments comprising molecules with carbonyl and hydroxyl groups. Glucose, for example, when broken into retro-aldol fragments yields glycolaldehyde.

It is well known in the art of chemicals manufacturing that catalysts may be described as homogeneous or heterogeneous, the former being those catalysts which exist and operate in the same phase as the reactants, while the latter are those that do not.

Typically, heterogeneous catalysts may be categorised into two broad groups. One group comprise the supported catalytic compositions where the catalytically active component is attached to a solid support, such as silica, alumina, zirconia, activated carbon or zeolites. Typically these are either mixed with the reactants of the process they catalyse, or they may be fixed or restrained within a reaction vessel and the reactants passed through it, or over it. The other group comprise catalytic compositions where the catalytically active component is unsupported, i.e. it is not attached, to a solid support, an example of this group is the Raney-metal group of catalysts. An example of a Raney-metal catalyst is Raney-nickel, which is a fine-grained solid, composed mostly of nickel derived from a nickel-aluminium alloy. The advantage of heterogeneous catalysts is that they can be retained in the reactor vessel during the process of extracting the unreacted reactants and the products from the reactor vessel, giving the operator the capability of using the same batch of catalysts many times over. However, the disadvantage of heterogeneous catalysts is that over time their activity declines, for reasons such as the loss, or leaching, of the catalytically active component from its support, or because the access of the reactants to the catalytically active component is hindered due to the irreversible deposition of insoluble debris on the catalyst's support. As their activity declines, catalysts need to be replaced, and for heterogeneous catalysts this inevitably requires the process that they catalyse to be stopped, and the reactor vessel to be opened up, to replace the deactivated catalyst with a fresh batch. Such down-time is costly to the operators of the process, as during such time no products can be produced, and such a labour-intensive operations have cost implications.

A further complication of using heterogeneous catalysts is that the process of preparing the catalyst, and in particular the process of immobilising catalytically active components onto a solid support in a way that gives maximum catalytic activity can be difficult and time consuming.

Homogeneous catalysts are typically unsupported and operate in the same phase as the reactants of the reaction they catalyse. Therefore their preparation does not require any step(s) for immobilising the catalytically active components onto a solid support, and their addition to, and mixing with, the reactants of the reaction they catalyse is much easier. However, separation of the catalyst from the reactants becomes more difficult, and in some cases not possible. This means that, in general, homogeneous catalysts either require to be replenished more often than heterogeneous catalysts, and/or additional steps and hardware are required in the process to remove the catalyst from the reactants and reaction products, with an obvious impact on the overall economy of the processes that they catalyse.

Regarding the two-step continuous process of making glycols from saccharide-containing feedstock, as described in WO2015028398, the activities and robustness of the at least two catalytic components, each of which is typically a heterogeneous catalyst, can vary with respect to each other, and therefore if the activity of any one of them declines sooner than the activity of the other, the process of glycol production will not go to completion, forcing the operators to stop the process to recharge one or both of the catalysts. Alternatively, breakdown components of one of the two catalytic components may adversely affect the other's activity. Again in such a case, the operators of the process are forced to stop the process to recharge one or both of the catalysts. A particular problem is caused by the catalyst component with retro-aldol catalytic capabilities, as over time it degrades and components leach from it. In particular, insoluble tungsten and molybdenum compounds and complexes are formed with the reactants in the reactor vessel over time. This problem is compounded by the deposition of organic degradation products, sintering of metal particles. Such insoluble matter attach to and clog up the surface of the catalyst component with hydrogenation capability, especially if such catalyst component comprises porous solid support and/or is unsupported but nevertheless has a porous surface topology (such as Raney-nickel). Further, the catalyst component with hydrogenation capability may also be poisoned by sulphur or other causes.

Therefore, it would be an advantage to prolong reactor runtimes by, for example, being able to supplement the hydrogenation activity in the reactor vessel without stopping and opening up the reactor vessel, simply by, for example, the addition to the reactor vessel of a solution of a hydrogenation catalyst precursor.

### SUMMARY OF THE INVENTION

The present invention concerns a process for the preparation of glycols from a saccharide-containing feedstock comprising the steps of: (a) preparing a reaction mixture in a reactor vessel comprising the saccharide-containing feedstock, a solvent, a catalyst component with retro-aldol catalytic capabilities and a first hydrogenation catalyst comprising an element selected from groups 8, 9 and 10 of the periodic table; (b) supplying hydrogen gas into the reaction mixture in the reactor vessel; (c) monitoring the hydrogenation activity in the reactor vessel; (d) when the hydrogenation activity declines, supplying into the reaction mixture in the reactor vessel a catalyst precursor comprising one or more elements selected from groups 8, 9, 10 and 11 of the periodic table; and (e) converting the catalyst precursor in the presence of hydrogen in the reactor vessel to a second hydrogenation catalyst to supplement the declined hydrogenation activity in the reactor vessel.

### DESCRIPTION OF THE DRAWINGS

FIG. 1 is a graph showing the levels of a product (MEG) produced (“Product yield” in % wt) during runs of the process according to the present invention.

### DETAILED DESCRIPTION OF THE INVENTION

The hydrogenation step in the process for the production of glycols from a saccharide-containing feedstock as described in WO2015028398 may be carried out with a Raney-metal type catalyst, which is readily available and is relatively cheap. Said hydrogenation step can also be carried out with other supported hydrogenation catalysts comprising an element selected from groups 8, 9 and 10 of the periodic table (i.e. other than the second hydrogenation catalyst claimed herein). However, because the process described in WO2015028398 is carried out in a single reactor vessel in the presence of a catalyst component with retro-aldol catalytic capabilities, both the Raney-metal hydrogenation catalyst and the supported hydrogenation catalysts comprising an element selected from groups 8, 9 and 10 of the periodic table are prone to deactivation by the degradation products of the a catalyst component with retro-aldol catalytic capabilities.

The inventors of the present processes have surprisingly found that a catalyst precursor can be converted into a second hydrogenation catalyst for the production of glycols from a saccharide-containing feedstock by supplying the catalyst precursor into the reactor vessel where said glycol production is taking place (‘in situ’ formation). The inventors have also found that such in situ formation of the second hydrogenation catalyst can be used to prolong the hydrogenation activity of the glycol production process by supplementing the declining hydrogenation activity of the commonly available hydrogenation catalyst that is already in the reactor vessel. Crucially, this overcomes the need to stop the reaction and open up the reactor vessel to replace the inactive commonly available hydrogenation catalyst.

In the process of glycol preparation from a saccharide-containing feedstock, a reaction mixture comprising the saccharide-containing feedstock, a solvent, a catalyst component with retro-aldol catalytic capabilities and a first hydrogenation catalyst is prepared in a reactor vessel, and hydrogen gas is supplied to the reaction mixture in the reactor vessel while the reactor vessel is maintained at a temperature and a pressure. Under these conditions, the catalyst component with retro-aldol catalytic capabilities converts the sugars in the saccharide-containing feedstock into retro-aldol fragments comprising molecules with carbonyl and hydroxyl groups, and in the presence of hydrogen, the first hydrogenation catalyst converts the these aldol fragments into glycols.

The glycols produced by the process of the present invention are preferably 1,2-butanediol, MEG and MPG, and more preferably MEG and MPG, and most preferably MEG. The mass ratio of MEG to MPG glycols produced by the process of the present invention is preferably 5:1, more preferably 7:1 at 230° C. and 8 MPa.

The saccharide-containing feedstock for the process of the present invention comprises starch. It may also comprise one or further saccharides selected from the group consisting of monosaccharides, disaccharides, oligosaccharides and polysaccharides. Examples of suitable disaccharides include glucose, sucrose and mixtures thereof. Examples of suitable oligosaccharides and polysaccharides include cellulose, hemicelluloses, glycogen, chitin and mixtures thereof.

In one embodiment, the saccharide-containing feedstock for said processes is derived from corn.

Alternatively, the saccharide-containing feedstock may be derived from grains such as wheat or, barley, from rice and/or from root vegetables such as potatoes, cassava or sugar beet, or any combinations thereof. In another embodiment, a second generation biomass feed such as lignocellulosic biomass, for example corn stover, straw, sugar cane bagasse or energy crops like Miscanthus or sweet sorghum and wood chips, can be used as, or can be part of, the saccharide-containing feedstock.

A pre-treatment step may be applied to remove particulates and other unwanted insoluble matter, or to render the carbohydrates accessible for hydrolysis and/or other intended conversions.

If required, further pre-treatment methods may be applied in order to produce the saccharide-containing feedstock suitable for use in the present invention. One or more such methods may be selected from the group including, but not limited to, sizing, drying, milling, hot water treatment, steam treatment, hydrolysis, pyrolysis, thermal treatment, chemical treatment, biological treatment, saccharification, fermentation and solids removal.

After the pre-treatment, the treated feedstock stream is suitably converted into a solution, a suspension or a slurry in a solvent.

The solvent may be water, or a C1 to C6 alcohol or polyalcohol, or mixtures thereof. Suitably C1 to C6 alcohols include methanol, ethanol, 1-propanol and isopropanol. Suitably polyalcohols include glycols, particularly products of the hydrogenation reaction, glycerol, erythritol, threitol, sorbitol, 1,2-hexanediol and mixtures thereof. More suitably, the poly alcohol may be glycerol or 1,2-hexanediol. Preferably, the solvent is water.

The concentration of the saccharide-containing feedstock as a solution in the solvent supplied to the reactor vessel is at most at 80% wt., more preferably at most at 60% wt. and more preferably at most at 45% wt. The concentration of the saccharide-containing feedstock as a solution in the solvent supplied to the reactor vessel is at least 5% wt., preferably at least 20% wt. and more preferably at least 35% wt.

The process for the preparation of glycols from a saccharide-containing feedstock requires at least two catalytic components. The first of these is a catalyst component with retro-aldol catalytic capabilities as described in patent application WO2015028398. The role of this catalyst in the glycol production process is to generate retro-aldol fragments comprising molecules with carbonyl and hydroxyl groups from the sugars in the saccharide-containing feedstock, so that the first hydrogenation catalyst can convert the retro-aldol fragments to glycols.

Preferably, the active catalytic components of the catalyst component with retro-aldol catalytic capabilities comprises of one or more compound, complex or elemental material comprising tungsten, molybdenum, vanadium, niobium, chromium, titanium or zirconium. More preferably the active catalytic components of the catalyst component with retro-aldol catalytic capabilities comprises of one or more material selected from the list consisting of tungstic acid, molybdic acid, ammonium tungstate, ammonium metatungstate, ammonium paratungstate, sodium metatungstate, sodium phosphotungstate, tungstate compounds comprising at least one Group I or II element, metatungstate compounds comprising at least one Group I or II element, paratungstate compounds comprising at least one Group I or II element, phosphotungstate compounds comprising at least one Group I or II element, heteropoly compounds of tungsten, heteropoly compounds of molybdenum, tungsten oxides, molybdenum oxides, vanadium oxides, metavanadates, chromium oxides, chromium sulphate, titanium ethoxide, zirconium acetate, zirconium carbonate, zirconium hydroxide, niobium oxides, niobium ethoxide, and combinations thereof. The metal component is in a form other than a carbide, nitride, or phosphide. Preferably, the second active catalyst component comprises one or more compound, complex or elemental material selected from those containing tungsten or molybdenum.

In one embodiment, the active catalytic components of the catalyst component with retro-aldol catalytic capabilities is supported on a solid support, and operates as a heterogeneous catalyst. The solid supports may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

In another embodiment, the active catalytic component of the catalyst component with retro-aldol catalytic capabilities is unsupported, and operates as a homogeneous catalyst. Preferably, in this embodiment the active catalytic components of the catalyst component with retro-aldol catalytic capabilities is metatungstate, which is delivered into the reactor vessel as an aqueous solution of sodium metatungstate.

The first hydrogenation catalyst comprises an element selected from groups 8, 9 and 10 of the periodic table. In one embodiment the first hydrogenation catalyst is a Raney-metal type catalyst, and preferably Raney-nickel catalyst. In another embodiment, the first hydrogenation catalyst comprises an element selected from groups 8, 9 and 10 of the periodic table supported on a solid support, such as ruthenium supported on activated carbon. The solid support may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

The catalyst precursor is a metal salt or a metal complex. In one embodiment, the catalyst precursor comprises a cation of an element selected from chromium and groups 8, 9, 10 and 11 of the periodic table. Preferably, the cation is of an element selected from the group consisting of chromium, iron, ruthenium, cobalt, rhodium, iridium, nickel, palladium, platinum and copper. More preferably the cation is of an element selected from the group comprising nickel, cobalt and ruthenium. Most preferably, the catalyst precursor comprises a ruthenium cation. In another embodiment, the catalyst precursor comprises a mixture of cations of more than one element selected from chromium and groups 8, 9, 10 and 11 of the periodic table. Preferably, the cations are of elements selected from the group consisting of chromium, iron, ruthenium, cobalt, rhodium, iridium, nickel, palladium, platinum and copper. Suitable examples of such mixture of cations may be a combination of nickel with palladium, or a combination of palladium with platinum, or a combination of nickel with ruthenium.

The catalyst precursor is a metal salt or a metal complex. In one embodiment, the catalyst precursor comprises an anion selected from the group consisting of inorganic anions and organic anions, preferably anions of carboxylic acids. In the case of both the organic and the inorganic anions, the anion must form a salt or a metal complex with the cations listed above, which is soluble in a mixture comprising the saccharide-containing feedstock, the solvent and the glycols. Preferably, the anion is selected from oxalate, acetate, propionate, lactate, glycolate, stearate, acetylacetonate, nitrate, chloride, bromide, iodide or sulphate. More preferably, the anion is selected from acetate, acetylacetonate or nitrate. Even more preferably, the anion is selected from acetate and acetylacetonate, and most preferably, the anion is acetylacetonate. In the embodiment where the catalyst precursor comprises more than one cation, the anion of each of the metal salts or metal complexes may be any one of the anions listed above, with the proviso that each metal salt or each metal complex must be soluble in a mixture comprising the saccharide-containing feedstock, the solvent and the glycols.

The catalyst precursor is preferably supplied to the reactor vessel as a solution in a solvent. Preferably, such solvent is water and/or a solution of glycols in water and/or the product stream from the reactor vessel used for the process of producing glycols described herein.

The solution of the catalyst precursor is preferably pumped into the reactor vessel, and mixed together with the reactor vessel contents.

Suitable reactor vessels that can be used in the process of the preparation of glycols from a saccharide-containing feedstock include continuous stirred tank reactors (CSTR), plug-flow reactors, slurry reactors, ebullated bed reactors, jet flow reactors, mechanically agitated reactors, bubble columns, such as slurry bubble columns and external recycle loop reactors. The use of these reactor vessels allows dilution of the reaction mixture to an extent that provides high degrees of selectivity to the desired glycol product (mainly ethylene and propylene glycols). In one embodiment, there is a single reactor vessel, which is preferably a CSTR.

There may be more than one reactor vessel used to carry out the process of the present invention. The more than one reactor vessels may be arranged in series, or may be arranged in parallel with respect to each other. In a further embodiment, two reactor vessels arranged in series, preferably the first reactor vessel is a CSTR, the output of which is supplied to a second reactor vessel, which is a plug-flow reactor vessel. The advantage provided by such two reactor vessel embodiment is that the retro-aldol fragments produced in the CSTR have a further opportunity to undergo hydrogenation in the second reactor, thereby increasing the glycol yield of the process. The second reactor vessel, which is a plug-flow reactor vessel, is suitably a fixed-bed type reactor.

Irrespective of whether there is a single reactor vessel or there are two reactor vessels, the catalyst component with retro-aldol catalytic capabilities is supplied preferably into the CSTR only. The weight ratio of the catalyst component with retro-aldol catalytic capabilities (based on the amount of metal in said composition) to the saccharide-containing feedstock is suitably in the range of from 1:100 to 1:1000.

The first hydrogenation catalyst may be either a Raney-metal type hydrogenation catalyst, or a supported hydrogenation catalyst comprising an element selected from groups 8, 9 and 10 of the periodic table.

In the embodiment where there is a CSTR only, if Raney-Nickel is chosen as the first hydrogenation catalyst, the quantity of Raney-nickel supplied to the CSTR is in a range of from 0.01 g metal per L reactor volume to 40 g metal per L reactor volume. Alternatively if a supported hydrogenation catalyst comprising an element selected from groups 8, 9 and 10 of the periodic table is chosen as the first hydrogenation catalyst, the maximum quantity supplied to the CSTR is about 10% volume in 90% volume liquid, which translates to about 4% weight.

In the embodiment with a CSTR followed by a plug-flow reactor arranged in series, the quantity of the first hydrogenation catalyst supplied to the CSTR is the same as stated in the preceding paragraph, and the quantity supplied to the plug-flow reactor vessel is typically 60% reactor vessel volume.

Preferably, the process of the present reaction takes place in the absence of air or oxygen. In order to achieve this, it is preferable that the atmosphere in the reactor vessel is evacuated after loading of any initial reactor vessel contents and before the reaction starts, and initially replaced with nitrogen gas. There may be more than one such nitrogen replacement step before the nitrogen gas is removed from the reactor vessel and replaced with hydrogen gas.

The process of the present invention takes place in the presence of hydrogen. In the embodiment where there is a single reactor vessel, hydrogen gas is supplied into the reactor vessel at a pressure of at least 1 MPa, preferably at least 2 MPa, more preferably at least 3 MPa. Hydrogen gas is supplied into the reactor vessel at a pressure of at most 13 MPa, preferably at most 10 MPa, more preferably at most 8 MPa. In the embodiment where there are two reactor vessels arranged in series, hydrogen is supplied in to the CSTR at the same pressure range as for the single reactor (see above), and optionally hydrogen may also be supplied into the plug-flow reactor vessel. If hydrogen is supplied into the plug-flow reactor vessel, it is supplied at the same pressure range as for the single reactor (see above).

In the embodiment where there is a single reactor vessel, the reaction temperature in the reactor vessel is suitably at least 130° C., preferably at least 150° C., more preferably at least 170° C., most preferably at least 190° C. In such embodiment, the temperature in the reactor vessel is suitably at most 300° C., preferably at most 280° C., more preferably at most 250° C., even more preferably at most 230° C. Preferably, the reactor vessel is heated to a temperature within these limits before addition of any reaction mixture, and is controlled at such a temperature to facilitate the completion of the reaction.

In the embodiment with a CSTR followed by a plug-flow reactor vessel arranged in series, the reaction temperature in the CSTR is suitably at least 130° C., preferably at least 150° C., more preferably at least 170° C., most preferably at least 190° C. The temperature in the reactor vessel is suitably at most 300° C., preferably at most 280° C., more preferably at most 250° C., even more preferably at most 230° C. In the embodiment with a CSTR followed by a plug-flow reactor vessel arranged in series, the reaction temperature in the plug-flow reactor vessel is suitably at least 50° C., preferably at least 60° C., more preferably at least 80° C., most preferably at least 90° C. The temperature in such reactor vessel is suitably at most 250° C., preferably at most 180° C., more preferably at most 150° C., even more preferably at most 120° C. Preferably, each reactor vessel is heated to a temperature within these limits before addition of any reaction mixture, and is controlled at such a temperature to facilitate the completion of the reaction.

The pressure in the reactor vessel (if there is only one reactor vessel), or the reactor vessels (if there are more than one reactor vessel), in which the reaction mixture is contacted with hydrogen in the presence of the first hydrogenation catalyst composition described herein is suitably at least 3 MPa, preferably at least 5 MPa, more preferably at least 7 MPa. The pressure in the reactor vessel, or the reactor vessels, is suitably at most 12 MPa, preferably at most 10 MPa, more preferably at most 8 MPa. Preferably, the reactor vessel is pressurised to a pressure within these limits by addition of hydrogen before addition of any reaction mixture and is maintained at such a pressure until all reaction is complete through on-going addition of hydrogen. In the embodiment where there are two reactor vessels arranged in series, a pressure differential in the range of from 0.1 MPa to 0.5 MPa exists across the plug-flow reactor vessel to assist the flow of the liquid phase through the plug-flow reactor vessel.

Irrespective of whether there is a single reactor vessel or there are two reactor vessels, in the process of the present invention the residence time of the reaction mixture in each reactor vessel is suitably at least 1 minute, preferably at least 2 minutes, more preferably at least 5 minutes. Suitably, the residence time of the reaction mixture in each reactor vessel is no more than 5 hours, preferably no more than 2 hours, more preferably no more than 1 hour.

The activity of the first hydrogenation catalyst can be monitored in a number of ways by measuring certain indications. For example, decline in product yield (e.g. MEG levels), decline in the formation of sugar alcohols like glycerin, erythritol, threitol and sorbitol, decline in pH due to formation of increased amounts of organic acids, increase in the levels of hydroxyketones, 2,3-butanediol and 2,3-pentanediol, increase in the levels of C3, C4 and C6 components relative to C2, are all indications of a decline in hydrogenation activity. One or more of these indications may be monitored at any one time. In one embodiment, the levels of hydroxyketones, such as hydroxyacetone or 1-hydroxy-2-butanone, exiting CSTR is monitored. In another embodiment, the level of glycerol exiting the plug-flow reactor vessel is monitored. A level of hydroxyketones relative to glucose of above 1% wt., and a level of glycerol relative to glucose of below 1% wt. are both indications that the hydrogenation reaction catalysed by the first hydrogenation catalyst has declined. Thus these values are a threshold, crossing of which indicate that the hydrogenation activity of the process needs to be increased, and this can the done by the supply of a quantity of the catalyst precursor to the reactor vessel(s) one or more times as needed. In the presence of hydrogen in the reactor vessel(s), the supplied catalyst precursor is converted into the second hydrogenation catalyst, thereby providing supplementary hydrogenation catalytic activity to the reactor vessel(s).

Irrespective of whether there is a single reactor vessel or there are two reactor vessels, the quantity of catalyst precursor supplied to each reactor vessel (in units of g metal per L reactor volume in each case) preferably at least at 0.01, more preferably at least at 0.1, even more preferably at least at 1 and most preferably at least 8. In such embodiment, the catalyst precursor is supplied to each reactor vessel (in units of g metal per L reactor volume in each case) preferably at most at 20, more preferably at most at 15, even more preferably at most at 12 and most preferably at most at 10.

In one embodiment, the catalyst precursor comprises ruthenium, which is supplied to each reactor vessel (in units of g metal per L reactor volume in each case) preferably at least at 0.01, more preferably at least at 0.1, even more preferably at least at 0.5. In such embodiment, the catalyst precursor comprising ruthenium is supplied to each reactor vessel (in units of g metal per L reactor volume in each case) preferably at most at 10, more preferably at most at 5, even more preferably at most at 2.

In another embodiment, the catalyst precursor comprises nickel, which is supplied to each reactor vessel (in units of g metal per L reactor volume in each case) preferably at least at 0.1, more preferably at least at 1, even more preferably at least at 5. In such embodiment, the catalyst precursor comprising nickel is supplied to each reactor vessel (in units of g metal per L reactor volume in each case) preferably at most at 20, preferably at most at 15, even more preferably at most at 10.

The inventors of the present invention believe that the surface topology of the micron-sized particles is smooth and does not contain any significant pores. The inventors of the present invention have found that such surface topology is resistant to insoluble compounds of tungsten sticking to it, and therefore its catalytic activity is unaffected. This allows the second hydrogenation catalyst to be used in the presence of a catalyst component with retro-aldol catalytic capabilities.

The present invention therefore provides the means of producing glycols from saccharide-containing feedstock using cheaper hydrogenation catalysts for as long as possible, then, without stopping or opening up the reactor vessel, supplementing the hydrogenation activity by converting a catalyst precursor, in the reactor vessel whilst the glycol preparation reaction is going on, to a second hydrogenation catalyst which is resistant to such insoluble degradation products. Because the level of the hydrogenation activity can be monitored, such supplementing can be carried out in incremental steps, thereby minimising the amount of the expensive and/or rare transition metals required for the catalyst precursor. Further, the combination of the ease of supplying the catalyst precursor to the reactor vessel, the simple step of the conversion of the catalytic precursor to the second hydrogenation catalyst in the reactor vessel, and the resistance of the resultant second hydrogenation catalyst to deactivation by insoluble degradation products generated by the catalyst component with retro-aldol catalytic capabilities all overcome the need for expensive and complicated reactor setup.

The present invention is further illustrated in the following Examples.

### EXAMPLES

### Comparative Example

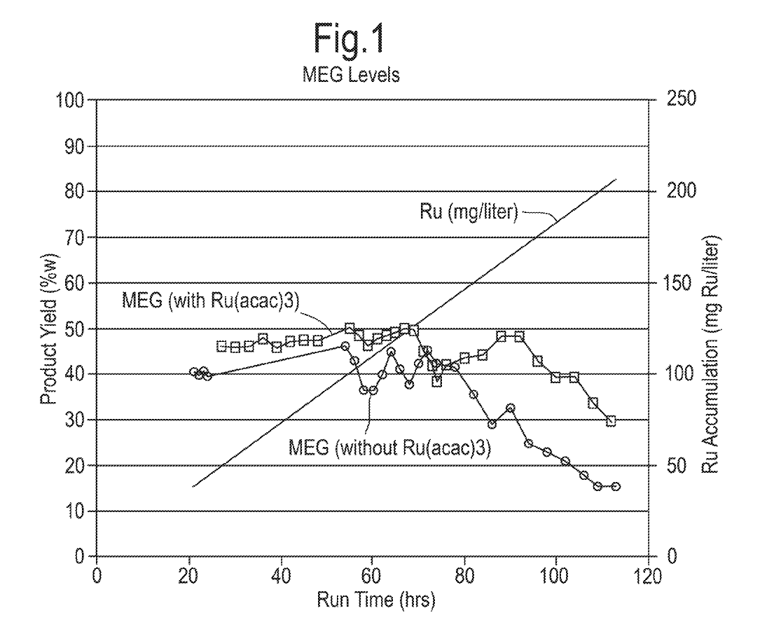

A 100 ml Hastelloy C22 reactor (Premex), equipped with a mechanical hollow-shaft gas stirrer, two liquid feed entries, one gas feed entry and a 5 micron filter connected to a gas/liquid discharge tube, was loaded with 41.5 g water and 3.5 g Raney-nickel, closed, pressurized with nitrogen to 90 barg and flushed with nitrogen at a rate of 9 liter STP/h for 10 min to replace air, prior to feeding hydrogen at a rate of 9 liter STP/h. Stirring is initiated at a rate of 1200 rpm and the temperature is raised to 230° C. while water is fed at a rate of 44 ml/h for three days. The liquid hold-up in the reactor is 50 ml on average. The liquid feed is switched from water to a solution containing 10% wt glucose, 2322 ppmw NaHCO₃ and 3800 ppmw sodium metatungstate at a rate of 44.2 ml/h, which is the start of the run time. The liquid, obtained after gas/liquid separation at room temperature, is analysed at regular time intervals for a period of 115 hours. Glucose conversions are 99.6% or higher during the experiment. During the first 76 hours an average MEG yield of about 40% wt is obtained, after which a gradual decline in MEG yield is observed during the subsequent period of 40 hours (FIG. 1). The initial sorbitol formation is 8.9% wt at 25 h run time, declining to 2.5% wt sorbitol at 69 h run time (Table 1), indicating a significant reduction in hydrogenation activity. Product yields are calculated as (weight of product)/(weight of glucose feed)*100%.

### Example 1

The procedure described in the Comparative Example is repeated, with the following differences: 2.5 g Raney-nickel is loaded, and finally two solutions are fed via two feed lines, the first being a water solution containing 20 ppmw Ru(acac)₃ at a rate of 10.3 ml/h and the second being a solution containing 13.5% wt glucose, 3000 ppmw NaHCO₃ and 4940 ppm sodium metatungstate at a rate of 33.0 ml/min. The averaged calculated feed composition is 4.8 ppmw Ru(acac)₃ (corresponding to 1.2 ppm Ru metal concentration), 10.3% wt glucose, 2270 ppmw NaHCO₃ and 3770 ppmw sodium metatungstate. Glucose conversions are 99.7% or higher during the experiment. MEG yields vary between 40% wt and 50% wt for more than 100 hours and are on average higher than in the Comparative Example, despite the lower amount of Raney-nickel applied, as depicted in FIG. 1. The initial hydrogenation activity is lower than in the Comparative Example, as indicated by an almost constant yield of sorbitol in the range of 2.5% wt-3.7% wt (Table 1). Apparently, 2.5 g Raney-nickel present in the current experiment exhibits a hydrogenation performance comparable to or superior to the 3.5 g Raney-nickel present in the Comparative Example, most probably due to the hydrogenation activity of accumulation ruthenium.

TABLE 1
Sugar Alcohol Yields, as Analysed by HPLC.

| Run | Runtime (hrs) | Sorbitol (% wt) |
|---|---|---|
| Comparative Example | 25 | 8.9 |
| | 69 | 2.5 |
| Example 1 | 26 | 2.5 |
| | 30 | 3.6 |
| | 51 | 2.9 |
| | 71 | 3.7 |

### DETAILED DESCRIPTION OF THE DRAWINGS

FIG. 1 is a graph showing the levels of a product (MEG) produced (“Product yield” in % wt) during runs of the process according to the present invention.

The continuous line that joins up the plotted diamond-shapes shows MEG levels during a run of the process according to the present invention, during which no catalyst precursor was supplied to the reactor vessel.

The continuous line that joins up the plotted square-shapes shows MEG levels during a run of the process according to the present invention, during which the catalyst precursor was supplied to the reactor vessel. During such run, the cumulative level of the catalyst precursor in the reactor vessel is indicated on the graph by the line which does not join up any geometric shapes.

During the first 76 hours of the run without any catalyst precursor supply to the reactor vessel, an average MEG yield of about 40% wt is obtained, however during the subsequent 40-hour period, a gradual decline in the MEG yield is observed (see the continuous line that joins up the plotted diamond-shapes). In comparison, the decline in the MEG yield is delayed during the run with the supply of the catalyst precursor to the reactor vessel (see the continuous line that joins up the plotted square-shapes).

1. A process for the preparation of glycols from a saccharide-containing feedstock comprising the steps of:
(a) preparing a reaction mixture in a reactor vessel comprising the saccharide-containing feedstock, a solvent, a catalyst component with retro-aldol catalytic capabilities and a first hydrogenation catalyst comprising an element selected from groups 8, 9 and 10 of the periodic table;
(b) supplying hydrogen gas into the reaction mixture in the reactor vessel;
(c) monitoring the hydrogenation activity in the reactor vessel;
(d) when the hydrogenation activity declines, supplying into the reaction mixture in the reactor vessel a catalyst precursor comprising one or more elements selected from groups 8, 9, 10 and 11 of the periodic table; and
(e) converting the catalyst precursor in the presence of hydrogen in the reactor vessel to a second hydrogenation catalyst to supplement the declined hydrogenation activity in the reactor vessel.

2. The process according to claim 1 wherein the catalyst precursor comprises one or more cations selected from a group comprising an element selected from groups 8, 9, 10 and 11 of the periodic table.

3. The process according to claim 1, wherein the cation is selected from a group consisting of iron, ruthenium, cobalt, rhodium, nickel, palladium and platinum.

4. The process according to claim 1, wherein the catalyst precursor comprises an anion selected from a group consisting of carboxylates, acetylacetonate and inorganic anions, which in all cases forms a salt or a complex that is soluble in a solvent mixture comprising the saccharide-containing feedstock, the solvent and the glycols.

5. The process according to claim 1, wherein the catalyst precursor comprises acetylacetonate.

6. The process according to claim 1, wherein the catalyst precursor comprises ruthenium cations.

7. The process according to claim 1, wherein the first hydrogenation catalyst is Raney-nickel.

8. A process according to claim 1, wherein the retro-aldol catalyst comprises tungsten.

9. A process according to claim 1, wherein the glycols comprise ethylene glycol and 1, 2-propylene glycol.

10. A process according to claim 1, wherein the saccharide-containing feedstock comprises one or more saccharide selected from the group comprising glucose, sucrose and starch.

11. A process according to claim 1, wherein the solvent is water, or a C1, C2, C3, C4, C5 or a C6 alcohol or polyalcohol, or any combination of mixtures thereof.
